(19) United States
(12) Patent Application Publication
(10) Pub. No.: US 2023/0392102 A1
(43) Pub. Date: Dec. 7, 2023

(54) CELL CULTURE SYSTEM AND METHODS OF USING THE SAME

(71) Applicant: Iovance Biotherapeutics, Inc., San Carlos, CA (US)

(72) Inventors: Seth Wardell, Tampa, FL (US); Joseph James Wypych, Tracy, CA (US)

(21) Appl. No.: 18/249,380

(22) PCT Filed: Oct. 22, 2021
(86) PCT No.: PCT/US2021/056125

§ 371 Date: Apr. 17, 2023

Related U.S. Application Data
(60) Provisional application No. 63/104,481, filed on Oct. 22, 2020.

(51) Int. Cl. C12M 1/04; C12M 1/36; C12M 1/00

(52) U.S. Cl. CPC C12M 23/24; C12M 41/48; C12M 29/04; C12M 47/20

(57) ABSTRACT

A gas permeable cell culture device including a container body defining an interior volume, the container body having a semi-permeable membrane defining one end of the interior volume, a fitting defining an opposing end of the interior volume, and a cell transfer conduit. The cell transfer conduit having an open end positioned within the interior volume between the semi-permeable membrane and the fitting and a radial portion disposed within the interior volume and between the open end and opposing end of the container body.

### CROSS-REFERENCE TO RELATED APPLICATIONS

This application claims the benefit of U.S. Provisional Patent Application No. 63/104,481 filed Oct. 22, 2020 entitled “Cell Culture System And Methods Of Using The Same”, which is incorporated by reference herein in its entirety.

### FIELD OF THE INVENTION

The invention relates to a cell culture system and methods of using the same, and more particularly, to a cell culture system and method housing and culturing cells.

### BACKGROUND OF THE INVENTION

Treatment of bulky, refractory cancers using adoptive transfer of tumor infiltrating lymphocytes (TILs) represents a powerful approach to therapy for patients with poor prognoses. A large number of TILs are required for successful immunotherapy, and a robust and reliable process is needed for commercialization. Further, devices for culturing, storing, and transferring TILs is crucial. Specifically, closed systems for culturing and storing TILs is important to ensure that the TILs are adequately preserved.

### BRIEF SUMMARY OF THE INVENTION

In one embodiment, there is a gas permeable cell culture device including a container body defining an interior volume, the container body having a semi-permeable membrane defining one end of the interior volume, a fitting defining an opposing end of the interior volume, and a cell transfer conduit having an open end positioned within the interior volume between the semi-permeable membrane and the fitting, a radial portion disposed within the interior volume and between the open end and opposing end of the container body.

In some embodiments, the interior volume is configured to include a headspace and at least a portion of the radial portion is disposed in the headspace.

In some embodiments, the radial portion comprises a segment of the cell transfer conduit having a longitudinal axis with at least one finite radius of curvature.

In some embodiments, the cell transfer conduit has an internal diameter of approximately 0.125 inches. The cell transfer conduit may include an interior portion extending from the fitting to the open end, the interior portion having an inner wall radially disposed about a longitudinal axis wherein no segment of the longitudinal axis of the interior portion has a radius of curvature that is less than 30 centimeters. The cell transfer conduit may include a transfer portion in fluid communication with the radial portion and extending from the fitting. The fitting may include a plurality of apertures, at least one of the plurality of apertures configured to receive the transfer portion of the cell transfer conduit.

In some embodiments, the gas permeable cell culture device of further includes a gas inlet conduit extending through the fitting.

In some embodiments, the gas permeable cell culture device further includes an extraction conduit extending into the head space through the fitting.

In some embodiments, the gas permeable cell culture device further includes a feeding conduit extending into the head space through the fitting.

In some embodiments, the feeding conduit includes a transfer portion extending through the fitting.

In some embodiments, the gas inlet conduit includes a transfer portion extending through the fitting.

In some embodiments, the gas permeable cell culture device further includes machine readable indicia disposed on a surface of the gas permeable cell culture device. The machine readable indicia may be readable to indicate at least one of the parameters selected from the group consisting of: 1) information associated with an origin of cells disposed within the cell culture device; 2) time information associated with injection and/or extraction of cells with respect to the cell culture device; 3) information with respect to media disposed within or extracted from the cell culture device; 4) information associated with a patient donor of the cells disposed within the cell culture device; 5) identification and tracking of a chain of custody and/or chain of identity associated with cell culture device and/or the cells disposed within the cell culture device; 6) information associated with quality control data; 7) information associated with electronic batch records; 8) information associated with manufacturing database and/or enterprise resource planning; and 9) combinations thereof.

In some embodiments, the machine readable indicia is selected from a group consisting of bar coding, QR coding, RFID, magnetic strips, one or more photographs or text.

In some embodiments, the cell transfer conduit passes through a vertical sidewall of the fitting.

In some embodiments, the gas permeable cell culture device further includes at least one conduit passing through a horizontal top wall of the fitting and the cell transfer conduit passing through a vertical sidewall of the fitting.

In some embodiments, the gas permeable cell culture device further includes a separation membrane disposed between the fitting and the interior volume of the container.

In some embodiments, the separation membrane includes a plurality of apertures, at least one of the plurality of apertures configured to receive a portion of the cell transfer conduit. The separation membrane may be coupled to the fitting.

In some embodiments, the fitting is removably coupled to the container. The fitting may be fixedly coupled to the container.

In some embodiments, the cell transfer conduit includes at least one attachment piece.

In some embodiments, the gas inlet conduit is coupled to a sterile filter sized between 0.05 micrometers and 0.25 micrometers.

In some embodiments, the gas permeable cell culture device further includes a bottom fitting coupled to a bottom of the container. The bottom fitting may be adjacent to the semi-permeable membrane. The bottom fitting may be configured to be removably coupled to a tray. The tray may be configured to receive a plurality of bottom fittings.

In some embodiments, the cell transfer conduit is configured to be heat welded to seal the cell transfer conduit.

In some embodiments, the gas inlet conduit includes a radial portion disposed within the interior volume and between the open end and opposing end of the container body.

In some embodiments, the gas permeable cell culture device further includes a gas inlet conduit, a extraction conduit, and a feeding conduit, wherein any of the gas inlet conduit, the extraction conduit, or the feeding conduit includes a radial portion disposed within the interior volume and between the open end and opposing end of the container body.

In some embodiments, a ratio of a volume of the container to a surface area of the semi-permeable membrane is between 1 mL/cm² and 10 mL/cm².

Another embodiment of the present invention provides a method of using the gas permeable cell culture device of claim 1 for expanding tumor infiltrating lymphocytes (TILs) into a therapeutic population of TILs, the method including:
(a) optionally pre-treating a patient with a regimen comprising a kinase inhibitor or an ITK inhibitor;
(b) obtaining a first population of TILs from a tumor resected from a patient by dissecting a tumor sample resected from a patient into multiple tumor fragments;
(c) adding into the gas permeable cell culture device, through the cell transfer conduit, the multiple tumor fragments;
(d) performing a first expansion, by culturing the multiple tumor fragments of the first population of TILs in a cell culture medium disposed within the gas permeable cell culture device, the cell culture medium comprising IL-2, and optionally OKT-3, to produce a second population of TILs during an expansion period of fourteen days or less after initiation of the first expansion, wherein the second population of TILs is at least 50-fold greater in number than the first population of TILs, and the only added source of gas to the expanding TIL population during the expansion period is through the semi-permeable membrane;
(e) performing a second expansion by supplementing the cell culture medium of the second population of TILs with additional IL-2, optionally OKT-3, and antigen presenting cells (APCs), to produce a third population of TILs, wherein the second expansion is performed for a second expansion period of fourteen days or less, wherein the third population of TILs is a therapeutic population of TILs, wherein the second expansion is performed in a second gas-permeable cell culture device and the only added source of gas to the second expanding TIL population during the second expansion period is through a second gas permeable membrane in the second gas-permeable cell culture device;
(f) harvesting the therapeutic population of TILs obtained from step (e); and
(g) transferring the harvested TIL population from step (f) to an infusion bag.

In some embodiments, steps (c)-(f) are performed within a closed system wherein the only source of added gas to the closed system is through first and second gas permeable membranes.

In some embodiments, the method further includes the step of cryopreserving the harvested TIL population in the infusion bag using a cryopreservation process.

In some embodiments, the cryopreservation process is performed using a 1:1 ratio of harvested TIL population to cryopreservation media.

In some embodiments, the antigen-presenting cells are peripheral blood mononuclear cells (PBMCs). The PBMCs may be irradiated and allogeneic. The PBMCs may be added to the cell culture on any of days 9 through 14 of the second expansion period.

In some embodiments, the antigen-presenting cells are artificial antigen-presenting cells.

In some embodiments, the harvesting is performed using a membrane-based cell processing system. The harvesting in step (f) may be performed using a LOVO cell processing system.

In some embodiments, the multiple tumor fragments comprise about 4 to about 50 fragments, wherein each fragment has a volume of about 27 mm³.

In some embodiments, the multiple tumor fragments comprise about 30 to about 60 fragments with a total volume of about 1300 mm³ to about 1500 mm³.

In some embodiments, the multiple tumor fragments comprise about 50 fragments with a total volume of about 1350 mm³.

In some embodiments, the multiple tumor fragments comprise about 50 fragments with a total mass of about 1 gram to about 1.5 grams.

In some embodiments, the cell culture medium is provided in a container selected from the group consisting of a G-container and a Xuri cellbag.

In some embodiments, the cell culture medium in step (d) further comprises IL-15 and/or IL-21.

In some embodiments, the IL-2 concentration is about 10,000 IU/mL to about 5,000 IU/mL.

In some embodiments, the IL-15 concentration is about 500 IU/mL to about 100 IU/mL.

In some embodiments, the IL-21 concentration is about 20 IU/mL to about 0.5 IU/mL.

In some embodiments, the infusion bag in step (g) is a HypoThermosol-containing infusion bag.

In some embodiments, the cryopreservation media comprises dimethlysulfoxide (DMSO). The cryopreservation media may comprise 7% to 10% DMSO.

In some embodiments, the first expansion period in step (d) and the second expansion period in step (f) are each individually performed within a period of 10 days, 11 days, or 12 days. The first expansion period in step (d) and the second expansion period in step (f) may be each individually performed within a period of 11 days.

In some embodiments, steps (b) through (g) are performed within a period of about 10 days to about 22 days, about 20 days to about 22 days, about 15 days to about 20 days, about 10 days to about 20 days, about 10 days to about 15 days,

In some embodiments, steps (b) through (g) are performed in 22 days or less.

In some embodiments, steps (b) through (g) are performed in 20 days or less.

In some embodiments, steps (b) through (g) are performed in 15 days or less.

In some embodiments, steps (b) through (g) are performed in 10 days or less.

In some embodiments, steps (b) through (g) or steps (b) through (f) and cryopreservation are performed in 22 days or less.

In some embodiments, the therapeutic population of TILs harvested in step (f) comprises sufficient TILs for a therapeutically effective dosage of the TILs. The population of TILs sufficient for a therapeutically effective dosage may be from about 2.3×10¹⁰ to about 13.7×10¹⁰.

In some embodiments, steps (c) through (f) are performed in a single container, wherein performing steps (c) through (f) in a single container results in an increase in TIL yield per resected tumor as compared to performing steps (c) through (f) in more than one container.

In some embodiments, the antigen-presenting cells are added to the TILs during the second period in step (e) without opening the cell culture device.

In some embodiments, the third population of TILs in step (e) provides for increased efficacy, increased interferon-gamma production, increased polyclonality, increased average IP-10, and/or increased average MCP-1 when administered to a subject.

In some embodiments, the third population of TILs in step (e) provides for at least a five-fold or more interferon-gamma production when administered to a subject.

In some embodiments, the third population of TILs in step (e) is a therapeutic population of TILs that comprises an increased subpopulation of effector T cells and/or central memory T cells relative to the second population of TILs, wherein the effector T cells and/or central memory T cells in the therapeutic population of TILs exhibit one or more characteristics selected from the group consisting of expressing CD27+, expressing CD28+, longer telomeres, increased CD57 expression, and decreased CD56 expression relative to effector T cells, and/or central memory T cells obtained from the second population of cells.

In some embodiments, the effector T cells and/or central memory T cells obtained from the third population of TILs exhibit increased CD57 expression and decreased CD56 expression relative to effector T cells and/or central memory T cells obtained from the second population of cells.

In some embodiments, a risk of microbial contamination is reduced when the cell culture device is a closed system compared to when the cell culture device is an open system.

In some embodiments, the method further includes infusing the TILs from step (g) into a patient.

In some embodiments, the multiple tumor fragments comprise about 4 fragments.

Another embodiment of the present invention provides a cell culture device including a top end cap having a first aperture, a bottom end cap having a second aperture aligned with the first aperture, and one or more assemblies disposed between the top end cap and the bottom end cap, wherein each one of the one or more of assemblies includes a top support, a bottom support, and a membrane layer disposed between the top support and the bottom support.

The cell culture device of claim 78, further comprising a central bore disposed through a central axis of the one or more assemblies, wherein the central bore aligns with the first aperture and the second aperture.

In some embodiments, each one of the one or more assemblies is disposed on an offset plane.

In some embodiments, each one of the one or more assemblies includes a first end and a second end offset from the first end, the first end and second end being disposed along an outer perimeter of each one of the one or more assemblies.

In some embodiments, one of the one or more assemblies is configured to be stackable with another one of the one or more assemblies such that the first end of one of the one or more assemblies is aligned with the first end of another one of the one or more assemblies.

In some embodiments, a thickness of the first end is greater than a thickness of the second end.

In some embodiments, each one of the one or more assemblies is circular.

In some embodiments, the membrane layer of each one of the one or more assemblies is a media-impermeable membrane. The membrane layer of each one of the one or more assemblies may be a gas permeable membrane.

In some embodiments, the membrane layer of each one of the one or more assemblies is comprised of one or more of polydimethylsiloxane, copolymers, polyolefins, flouropolymers, fluorinated ethylene propylene (FEP), polyvinylchloride (PVC), ethytlene-vinyl acetate (EVA), and other polymers or copolymers.

In some embodiments, the membrane layer of each one of the one or more assemblies is comprised of polystyrene film.

In some embodiments, the membrane layer of each one of the one or more assemblies has a thickness between about 25 micrometers and 125 micrometers.

In some embodiments, the bottom support and/or the top support of each one of the one or more assemblies is comprised of polystyrene.

In some embodiments, the membrane layer is coupled to the top support via one of adhesive bonding, ultrasonic welding, or laser welding.

In some embodiments, the top support is coupled to the bottom support via one of adhesive bonding, ultrasonic welding, or laser welding.

In some embodiments, the cell culture device further includes a port formed between the top support and the bottom support to allow for gas exchange. The port may be disposed on an outer perimeter of each one of the one or more assemblies.

In some embodiments, the cell culture device further includes a housing and a housing cap coupled to the housing, wherein the one or more assemblies are disposed within the housing. The housing cap may include one or more openings and one or more conduits disposed within the one or more openings, each one of the one or more conduits extending from the one or more openings of the housing cap into the housing.

In some embodiments, at least one of the one or more conduits is in a spiral configuration within the housing cap prior to extending within the housing.

In some embodiments, each one of the one or more conduits has a different length.

In some embodiments, at least one of the one or more conduits includes an elbow joint disposed within the housing cap. Another embodiment of the present invention provides a single use syringe including IL-2 at a volume and biological activity sufficient to continuously expand T-cells for a predetermined period of time, the T-cells being injected into a source of cell culture media via an attachment device and the cell culture media is subsequently added to a bioreactor suitable for expansion of T-cells.

In some embodiments, the predetermined period of time is 5-11 days and the attachment device is a luer lock.

In some embodiments, the predetermined period of time is 5-11 days and the attachment device is a needleless injection site or other syringe coupler.

In some embodiments, the predetermined period of time is 5-11 days and the attachment device is a sterile connecting device.

In some embodiments, the predetermined period of time is 5-11 days and the attachment device is a luer lock connection to the bioreactor.

In some embodiments, the predetermined period of time is 5-11 days and the attachment device is a needleless injection site or other syringe coupler connection to the bioreactor.

In some embodiments, the predetermined period of time is 5-11 days and the attachment device is a sterile connecting device connection to the bioreactor.

In some embodiments, the predetermined period of time is 5-11 days and the attachment device is a luer lock and T-cells are pumped or gravity drained into the cell culture media.

In some embodiments, the predetermined period of time is 5-11 days and the attachment device is a needleless injection site or other syringe coupler and T-cells are pumped or gravity drained into the cell culture media.

In some embodiments, the predetermined period of time is 5-11 days and the attachment device is a sterile connecting device and T-cells are pumped or gravity drained into the cell culture media.

Yet another embodiment of the present invention provides a collapsible container including IL-2 at a volume and biological activity sufficient to continuously expand T-cells for a predetermined amount of time, the container connected via an attachment device and pumped or gravity drained into a bioreactor suitable for expansion of T-cells.

In some embodiments, the predetermined amount of time is 5-11 days and the attachment device is a luer lock.

In some embodiments, the predetermined amount of time is 5-11 days and the attachment device is a needleless injection site or other sterile coupler.

In some embodiments, the predetermined amount of time is 5-11 days and the attachment device is a sterile connecting device.

### BRIEF DESCRIPTION OF THE SEVERAL VIEWS OF THE DRAWINGS

The foregoing summary, as well as the following detailed description of embodiments of the system and apparatus of transforming financial data, will be better understood when read in conjunction with the appended drawings of an exemplary embodiment. It should be understood, however, that the invention is not limited to the precise arrangements and instrumentalities shown.

FIG. 1 is an exemplary cell culture device in accordance with one embodiment of the present invention;

FIG. 2 is a top view of the exemplary cell culture device of FIG. 1;

FIG. 3 is a cross-sectional view of the exemplary cell culture device of FIG. 2;

FIG. 4 is an exemplary spiral stack assembly in accordance with one embodiment of the present invention;

FIG. 5 is an exemplary assembly in accordance with one embodiment of the present invention;

FIG. 6 is a side view of the exemplary assembly of FIG. 5;

FIG. 7 is an exemplary set of assemblies in accordance with one embodiment of the present invention;

FIG. 8 is a top perspective view of the exemplary assembly of FIG. 5;

FIG. 9 is an exemplary method for use with a cell culture device in accordance with one embodiment of the present invention;

FIG. 10 is an exemplary delivery device in accordance with one embodiment of the present invention;

FIG. 11 is an adapter to be used with an exemplary delivery device in accordance with one embodiment of the present invention;

FIG. 12 is an adapter to be used with an exemplary delivery device in accordance with one embodiment of the present invention;

FIG. 13 is an exemplary delivery device in accordance with one embodiment of the present invention; and

FIGS. 14A and 14B are exemplary containers in accordance with embodiments of the present invention; and

FIGS. 15A-15D are exemplary containers in accordance with embodiments of the present invention.

### DETAILED DESCRIPTION OF THE INVENTION

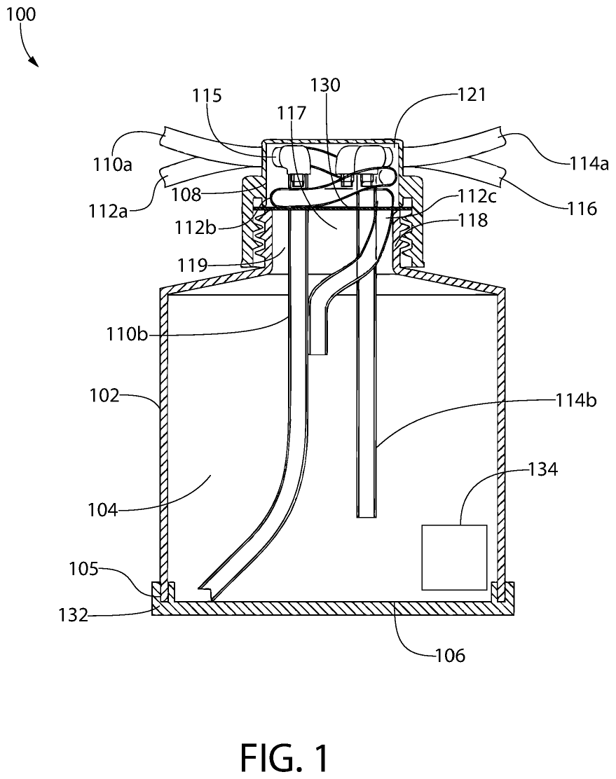

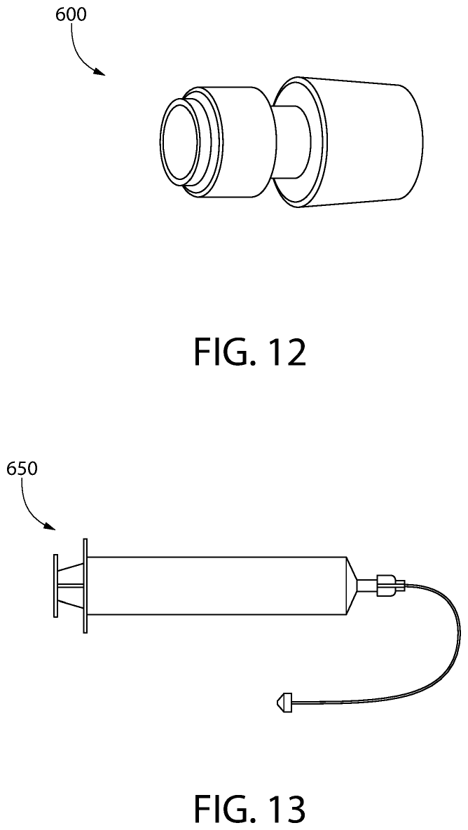

Exemplary embodiments of the present invention provide a cell culture device and methods of using the same. Embodiments of the present invention provide an exemplary cell culture device and a method of using the same as shown in FIGS. 1-15D. In use, the systems and methods presented herein may allow for the culturing, storage, transferring, and growth of biological material, such as tumor infiltrating lymphocytes (TILs).

Referring to FIGS. 1-3, cell culture device 100 may include container 102, fitting 108, and membrane 106. Container 102 may define an interior volume 104 and may include membrane 106 on one end of interior volume 104 and fitting 108 on the opposing end of interior volume 104. In some embodiments, membrane 106 may be a semi-permeable membrane. However, membrane 106 may be any type of membrane, such as a completely permeable membrane or non-permeable membrane. Although FIGS. 1-3 show container 102 being substantially cylindrical in shape, container 102 may be rectangular, hexagonal, pentagonal, octagonal, or any other shape desired. Container 102 may include open end 117. In some embodiments, open end 117 is opposite membrane 106. For example, membrane 106 may be disposed on the bottom of container 102 and open end 117 may be disposed at the top of container 102. Open end 117 may be disposed at top end 118 of container 102. In some embodiments, fitting 108 is coupled to the top of open end 117 and membrane 106 is coupled to bottom end 105 resulting in cell culture device 100 being a closed system. Cell culture device 100 being a closed system may prevent debris, bacteria, or other undesired particulates from entering container 102. For example, biological material, such as TILs may be added to cell culture device 100 and cell culture device 100 being a closed system may prevent contamination of the TILs. In some embodiments, fitting 108 is coupled to container 102 such that addition of air/materials is controlled. For example, the addition of air/materials may be controlled by via conduits coupled to fitting 108. In some embodiments, air/materials are added to container 102 via membrane 106.

In some embodiments, fitting 108 is coupled to container 102. Fitting 108 may be removably coupled to container 102 such that fitting 108 may be removed from container 102 by a user. In some embodiments, fitting 108 is threaded to allow fitting 108 to be coupled to container 102 and allow cell culture device 100 to be a closed system. However, fitting 108 may be fixedly coupled to container 102. Fitting 108 may be coupled to container 102 such that conduits disposed within and/or through fitting 108 provide the main access to interior volume 104. In some embodiments, materials are introduced into container 102 through conduits fitting 108.

In practice, container 102 is configured to grow and culture organic material, such as TILs. In some embodiments, membrane 106 is configured to grow and propagate TILs. For example, membrane 106 may be configured allow for the proliferation and expansion of TILs. In some embodiment, container 102 is configured to hold 1000 mL of fluid volume. However, container 102 may be configured to hold from 50 mL to 5000 mL, from 150 mL to 2500 mL, from 250 mL to 2000 mL or from 500 mL to 1000 mL of fluid volume.

Referring to FIG. 1, cell culture device 100 may include membrane 106 coupled to bottom end 105 of container 102. Membrane 106 may be configured to allow cell culture device 100 to be a closed system. In some embodiments, membrane 106 has a surface area within container 102 of 100 cm². However, membrane 106 may have a surface area from 10 cm² to 200 cm², from 50 cm² to 175 cm², or from 100 cm² to 150 cm². In some embodiments, the ratio of the volume of container 102 to the surface area of membrane 106 within container 102 is from 1 mL/cm² to 25 mL/cm², from 5 mL/cm² to 20 mL/cm², or from 10 mL/cm² to 15 mL/cm².

In some embodiments, membrane 106 is sterile and configured to allow for the growth of cells, such as TILs. For example, membrane 106 may be configured to only allow certain molecules to pass through into interior volume 104. In some embodiments, membrane 106 is a gas permeable membrane. Membrane 106 may be configured to allow for the introduction of liquids, materials, and/or nutrients into container 102. For example, container 102 and membrane 106 may be placed on a nutrient-rich solution that may be absorbed through membrane 106 into container 102.

In some embodiments, cell culture device 100 includes bottom fitting 132 disposed at bottom end 105 of container 102. Bottom fitting 132 may be disposed adjacent membrane 106. In some embodiments, bottom fitting 132 includes membrane 106. Bottom fitting 132 may be configured to removably coupled cell culture device 100 to an object, such as a tray. In some embodiments, the tray is configured to receive and secure cell culture device 100 via bottom fitting 132. The tray may be configured to receive and secure more than one bottom fitting 132 thereby allowing the tray to receive and secure multiple cell culture devices 100. For example, the tray may be configured to receive and secuire multiple cell culture device 100. In some embodiments, tray is configured to receive and secure cell culture devices 100 of different sizes and configurations.

Referring to FIGS. 1-3, cell culture device 100 may include fitting 108. Fitting 108 may be secured to top end 118 of container 102 opposite membrane 106. Fitting 108 may include interior space 121, which may be space disposed within fitting 108. Fitting 108 and top end 118 may be threaded to allow fitting 108 to be securely coupled to top end 118 of container 102. Fitting 108 may include one or more apertures 115 configured to receive one or more conduits or tubes. In some embodiments, fitting 108 includes vertical sidewall 109 circumferentially disposed around fitting 108. One or more of the plurality of conduits may extend through vertical sidewall 109. For example, vertical sidewall 109 may include one or more apertures, each configured to receive a conduit. In some embodiments, one or more conduits may extend through vertical sidewall 109 through interior space 121 into container 102. In some embodiments, at least one conduit passes through horizontal top wall 111 of fitting 108. In some cell culture device 100 is configured facilitation the insertion of fluids and/or materials into the cell culture device 100 without exposing the interior of cell culture device 100 to atmosphere, directly or indirectly.

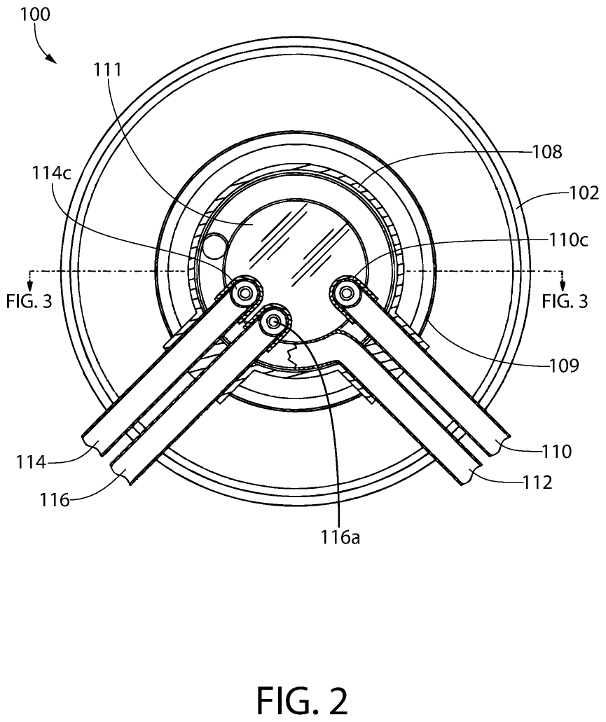

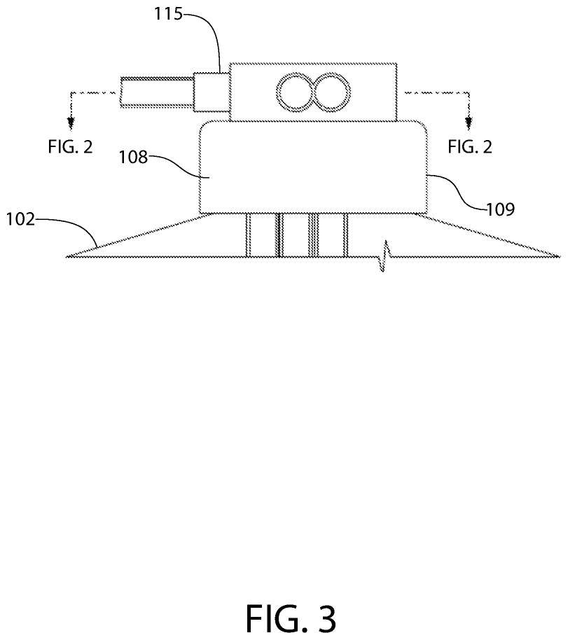

Referring to FIGS. 1-2, one of the plurality of conduits of cell culture device 100 may include transfer conduit 112, which may have an open end positioned within interior volume 104 between membrane 106 and fitting 108. Transfer conduit 112 may be a cell material transfer conduit configured to transfer fluids and/or materials into interior volume 104 and onto membrane 106. In some embodiments, transfer conduit 112 may include at least one of transfer portion 112a, radial portion 112b, and interior portion 112c. Radial portion 112b may be disposed between transfer portion 112a and interior portion 112c. In some embodiments, radial portion 112b is disposed within interior volume 104 and be between top end 118 and bottom end 105 of container 102. In some embodiments, transfer conduit 112 includes at least one attachment piece. For example, transfer conduit 112 may include a valve to control the transfer of fluids and/or materials into container 102. In some embodiments, a portion of transfer conduit 112 may be sealed. For example, transfer portion 112a may include an open end distal to fitting 108, which may be sealed, such as by heat welding. Sealing of transfer conduit 112 may prevent additional material from inadvertently entering interior volume 104. In some embodiments, transfer conduit 112 is configured to provide a conduit to transfer materials into container 102. Once materials have been transferred into container 102, transfer conduit 112 may be sealed to prevent inadvertent contamination or entrance of contaminants through transfer conduit 112.

In some embodiments, interior volume 104 includes headspace 119 and at least a portion of radial portion 112b is disposed within headspace 119. Headspace 119 may be disposed between interior space 121 and interior volume 104. Radial portion 112b may be a portion of transfer conduit 112 having a longitudinal axis with at least one finite radius of curvature. In some embodiments, transfer conduit 112 has an internal diameter of approximately 0.125 inches. However, transfer conduit 112 may have an internal diameter from 0.03125 inches to 5 inches, 0.0625 inches to 3 inches, 0.125 inches to 2 inches, or 0.25 inches to 1 inch.

In some embodiments, transfer conduit 112 includes interior portion 112c, which extends from fitting 108 to open end 117. In some embodiments, portion 112c includes an inner wall radially disposed about a longitudinal axis wherein no segment of the longitudinal axis of interior portion 112c has a radius of curvature. In some embodiments, the radius of curvature of the longitudinal axis of interior portion 112c is less than 30 centimeters. However, the radius of curvature of the longitudinal axis of interior portion 112c may be less than 100 centimetres, less than 75 centimeters, less than 50 centimeters, or less than 25 centimeters. In some embodiments, transfer conduit 112 includes transfer portion 112a in fluid communication with radial portion 112b and extending from one of the plurality of apertures 115 of fitting 108. For example, at least one of the plurality of apertures 115 of fitting 108 may be configured to receive transfer portion 112a. In some embodiments, transfer portion 112a is configured to pass through vertical sidewall 109.

Referring to FIGS. 1-2, one of the plurality of conduits of cell culture device 100 may include gas conduit 116. In some embodiments, Gas conduit 116 extends through fitting 108 and is configured to allow for exchange of gas between the environment and interior volume 104. In some embodiments, gas conduit 116 is coupled to a gas source to allow for the inflow of gas into interior volume 104. In some embodiments, gas conduit 116 is coupled to joint 116a. Joint 116a may be a joint disposed within fitting 108 and may be configured to couple gas conduit 116 to fitting 108. In some embodiments, joint 116a is an elbow joint to allow for fluids/material to flow from gas conduit 116 into interior volume 104. However, joint 116a may be any type of joint that allows for the fluids/materials to flow from gas conduit 116 into interior volume 104. In some embodiments, gas conduit 116 is configured to pass through vertical sidewall 109. Joint 116a may be configured to pass through vertical sidewall 109 and allow gas conduit 116 to also pass through vertical sidewall 109.

In some embodiments, gas conduit 116 is coupled to an attachment fitting. such as a sterile filter. The sterile filter may be from 0.02 μm to 1 μm, 0.03 μm to 0.75 μm, or 0.05 μm to 0.25 μm. In some embodiments, gas conduit 116 includes a coiled radial portion disposed within interior volume 104. The radial portion may be disposed between open end 117 and bottom end 105 of container 102. In some embodiments, the coiled radial portion is disposed within fitting 108.

In some embodiments, one of the plurality of conduits of cell culture device 100 includes extraction conduit 110. Extraction conduit 110 may include transfer portion 110a and extending portion 110b. In some embodiments, transfer portion 110a is coupled to fitting 108 and extending portion 110b extends into headspace 119 through fitting 108. Transfer portion 110a may extend through fitting 108. In some embodiments, extraction conduit 110 is coupled to joint 110c. Joint 110c may be a joint disposed within fitting 108 and may be configured to couple extraction conduit 110 to fitting 108. In some embodiments, joint 110c is an elbow joint to allow for fluids/material to flow from interior volume 104 out of container 102. However, joint 110c may be any type of joint to allow for fluids/material to flow from interior volume 104 out of container 102.

In some embodiments, extending portion 110b of extraction conduit 110 extends from headspace 119 to adjacent membrane 106. In some embodiments, extraction conduit 110 is configured to pass through vertical sidewall 109. In some embodiments, extraction conduit 110 includes a radial portion disposed within interior volume 104. The radial portion may be disposed between open end 117 and bottom end 105 of container 102.

In some embodiments, one of the plurality of conduits of cell culture device 100 may include feeding conduit 114. Feeding conduit 114 may include transfer portion 114a and extending feeding portion 114b. Transfer portion 114a may be coupled to fitting 108 and extending feeding portion 114b may extend into headspace 119 through fitting 108. Transfer portion 114a may extend through fitting 108. In some embodiments, transfer conduit 114 includes a radial portion disposed within interior volume 104. The radial portion may be disposed between open end 117 and bottom end 105 of container 102.

In some embodiments, feeding conduit 114 is coupled to joint 114c. Joint 114c may be a joint disposed within fitting 108 and may be configured to couple feeding conduit 114 to fitting 108. In some embodiments, joint 114c is an elbow joint to allow for fluids/material to flow into interior volume 104 of container 102. However, joint 114c may be any type of joint to allow for fluids/material to flow into interior volume 104 of container 102. In some embodiments, feeding conduit 114 is configured to pass through vertical sidewall 109. Joint 114c may be configured to pass through vertical sidewall 109 and allow feeding conduit 114 to also pass through vertical sidewall 109.

Referring to FIG. 1, fitting 108 may include separation membrane 130. Separation membrane 130 may be disposed between fitting 108 and interior volume 104. Separation membrane 130 may include a plurality of apertures. In some embodiments, at least one of the plurality of apertures is configured to receive a portion of at least one of the plurality of conduits. For example, separation membrane 130 may be configured to receive at least a portion of transfer conduit 112, extraction conduit 110, and/or feeding conduit 114. In some embodiments, separation membrane 130 is coupled to fitting 108. For example, separation membrane 130 may be coupled to fitting 108 adjacent to open end 117 of container 102 when fitting 108 is coupled to container 102.

Referring to FIG. 1, cell culture device 100 may include machine readable indicia 134. In some embodiments, machine readable indicia 134 is disposed on a surface of container 102. Machine readable indicia 134 may be bar coding, QR coding, RFID, magnetic strips, or one or more photographs or text. Machine readable indicia 134 may be readable and configured to indicate information associated with the origin of cells disposed within cell culture device 100. For example, machine readable indicia 134 may be configured to indicate the type of cells deposited within cell culture device 100, date that the cells were created, cultured, or deposited, or the source of the cells. Machine readable indicia 134 may be configured to indicate time information associated with injection and/or extraction of cells with respect to the cell culture device, information with respect to media disposed within or extracted from the cell culture device, information associated with the patient donor of the cells disposed within the cell culture device, identification and tracking of a chain of custody and/or chain of identity associated with cell culture device and/or the cells disposed within the cell culture device, information associated with quality control data, information associated with electronic batch records, and/or information associated with manufacturing database and/or enterprise resource planning.

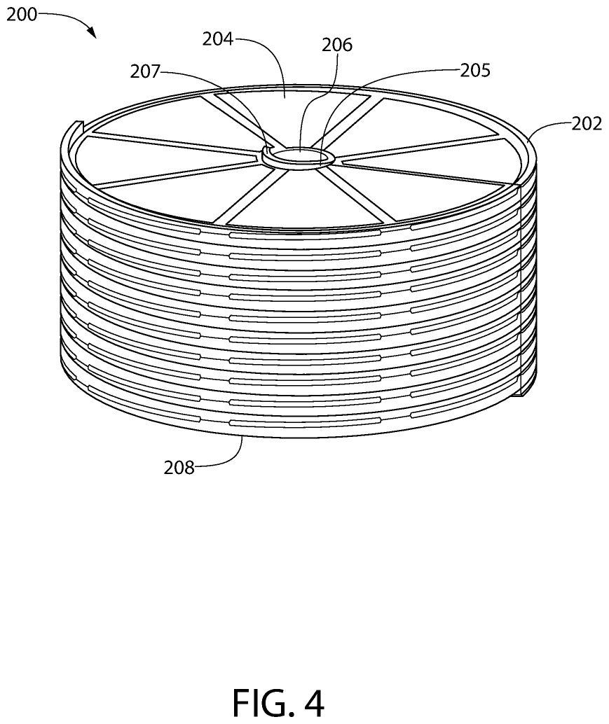

Referring to FIGS. 4-8, cell culture device 100 may include stack 200. Stack 200 may be disposed within cell culture device 100. For example, stack 200 may be disposed within interior volume 104 of cell culture device 100. Stack 200 may include top end cap 206 and bottom end cap 208, and may be comprised of a plurality of assemblies 202. Top end cap 206 may include first aperture 205 and bottom end cap 208 may include second aperture (not shown), which may be aligned with first aperture 205. In some embodiments, stack 200 may be configured to be disposed within assembly 202. Stack 200 may include central bore 207. Central bore 207 may be disposed between top end cap 206 and bottom end cap 208. In some embodiments, central bore 207 is disposed through a central axis of the plurality of assemblies 202 and may be aligned with first aperture 205.

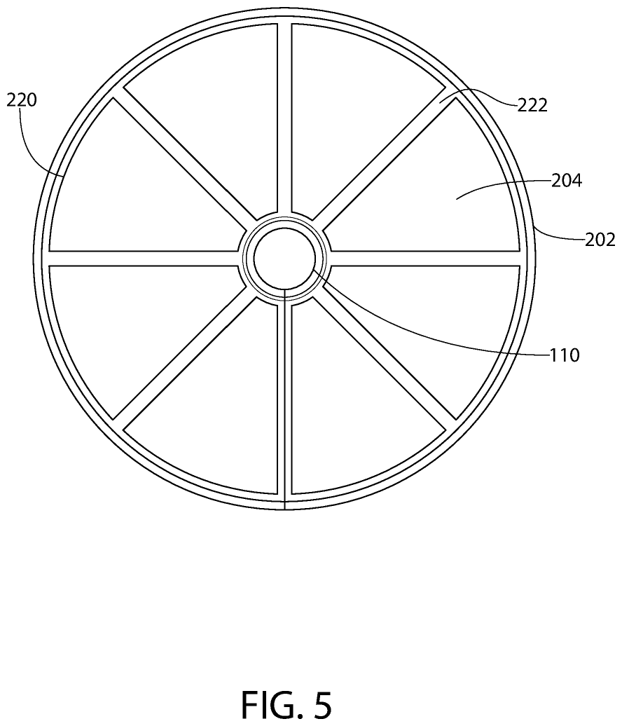

Referring to FIGS. 4 and 5, assemblies 202 may be configured to be stackable to form stack 200. For example, stack 200 may be comprised of 100 assemblies 202. However, stack 200 may be comprised of 2 to 500 assemblies, 50 to 300 assemblies, 75 to 250 assemblies, and 100 to 200 assemblies. In some embodiments, stack 200 may be comprised of a plurality of assemblies 202 disposed between top end cap 206 and bottom end cap 208. Assembly 202 may be substantially circular in shape and may have an outer diameter less than the diameter of container 102. However, assembly 202 may be square shaped, rectangular shaped, hexagonal shape, octagonal shaped, or any other shape desired.

In some embodiments, stack 200 may be configured to be disposed above membrane 106. For example, stack 200 may be disposed within container and may be disposed between membrane 106 and fitting 108. Stack 200 may be configured to sit above membrane 106 or rest on top of membrane 106. Cell culture device 100 having stack 200 may be configured to allow for the culturing and growth of a substantially more cells, such as TILs, compared to cell culture device 100 without stack 200. For example, each one of assemblies 202 of stack 200 may be configured to culture and grow substantially the same amount of cells as a single membrane 106. In practice, cell culture device 100 having stack 200 increases the amount of expansion of TILs compared to cell culture device 100 not having stack 200. Stack 200 allows for simultaneous expansion of multiple membranes housing TILs via assemblies 202.

Referring to FIG. 5, assembly 202 may include membrane 204. Membrane 204 may be substantially the same as membrane 106. For example, membrane 204 may be a semi-permeable membrane. In some embodiments, one or more membranes 204 may be disposed within each of assembly 202. Assembly 202 may include one or more sections 220. In some embodiments, each section 220 includes membrane 204. Assembly 202 may include a plurality of sections 220, each separated by separator 222. For example, assembly 202 may include eight sections 220 resulting in assembly 202 having eight membranes 204. However, assembly 202 may have between 1 and 20 sections, 5 and 15 sections, or 7 and 12 sections.

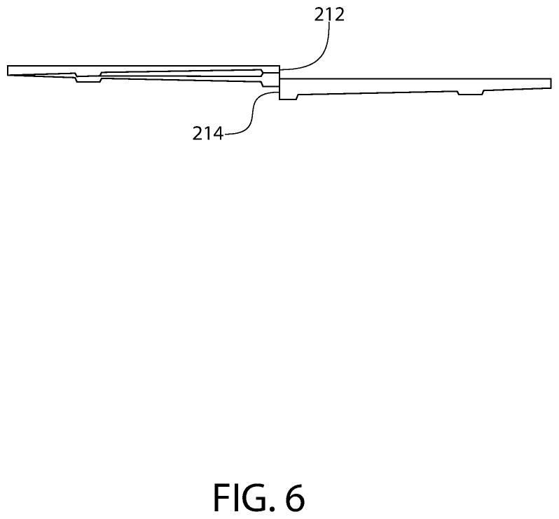

Referring to FIG. 6, assembly 202 may include first end 212 and second end 214. In some embodiments, first end 212 and second end 214 are disposed along the circumference of assembly 202. For example, first end 212 and second end 214 may be disposed along the outer perimeter of assembly 202. Second end 214 may have a thickness greater than first end 212. First end 212 and second end 214 may be offset from one another. For example, first end 212 may be along a first plane and second end 214 may be along a second plane, and the first plane and the second plane may be offset from one another. In some embodiments, the second plane of second end 214 may have a helix angle of approximately 1.3°. However, second end 214 may have a helix angle from 0° and 10°.

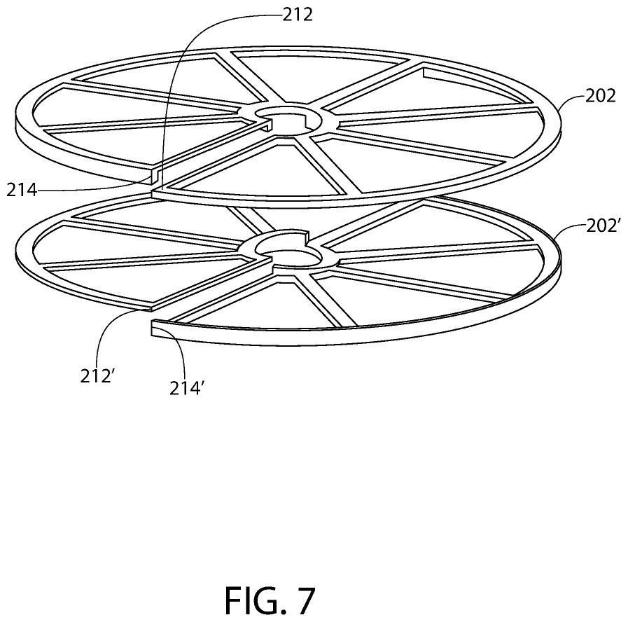

Referring to FIG. 7, to form stack 200, two assemblies 202, 202′ are stacked together such that first end 212 of assembly 202 is disposed adjacent to first end 212′ of another assembly 202′. For example, assembly 202 may be flipped along its horizontal axis such that first end 212 of assembly 202 can be aligned with first end 212′ of assembly 202′. Stack 200 may include a plurality of assemblies 202, which are stacked two at a time in configuration detailed in FIG. 7. In alternative embodiments, second end 214 of one assembly 202 may adjacent to or coupled to first end 212 of another assembly 202 when assemblies 202 are stacked together to form stack 200.

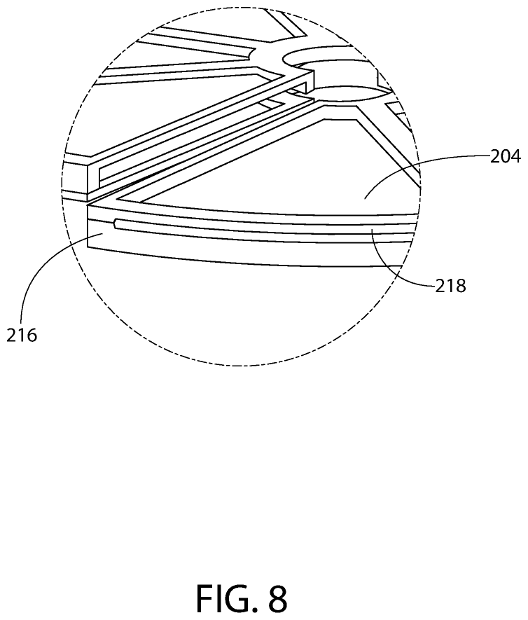

Referring to FIG. 8, assembly 202 may be comprised of top support 218 and bottom support 216. Top support 218 and/or bottom support 216 may be comprised of polystyrene. However, top support 218 and bottom support 216 may be comprised of polyurethane, polycarbonate, or any other type of polymer. In some embodiments, membrane 204 may be disposed and secured between top support 218 and bottom support 216. Membrane 204 may be a media-impermeable membrane substantially similar to membrane 106. In some embodiments, membrane 204 is a gas permeable membrane. Membrane 204 may be comprised of one or more of polydimethylsiloxane, copolymers, polyolefins, flouropolymers, fluorinated ethylene propylene (FEP), polyvinylchloride (PVC), ethylene-vinyl acetate (EVA), and other polymers or copolymers. In some embodiments, membrane 204 is comprised of polystyrene film. In some embodiments, membrane 204 has a thickness from about 25 μm to about 125 μm. However, membrane 204 may have a thickness from about 5 μm to about 250 μm, about 25 μm to about 200 m, or about 50 μm to about 150 μm.

Membrane 204 may be coupled to top support 218 and/or bottom support 216 via adhesive bonding, ultrasonic welding, or laser welding. In some embodiments, top support 218 is coupled to bottom support 216 by one of adhesive bonding, ultrasonic welding, or laser welding.

In some embodiments, assembly 202 includes a port, which is be formed between top support 218 and bottom support 216. The port may be configured to allow for gas exchange within assembly 202. In some embodiments, the port is disposed on an outer perimeter of assembly 202.

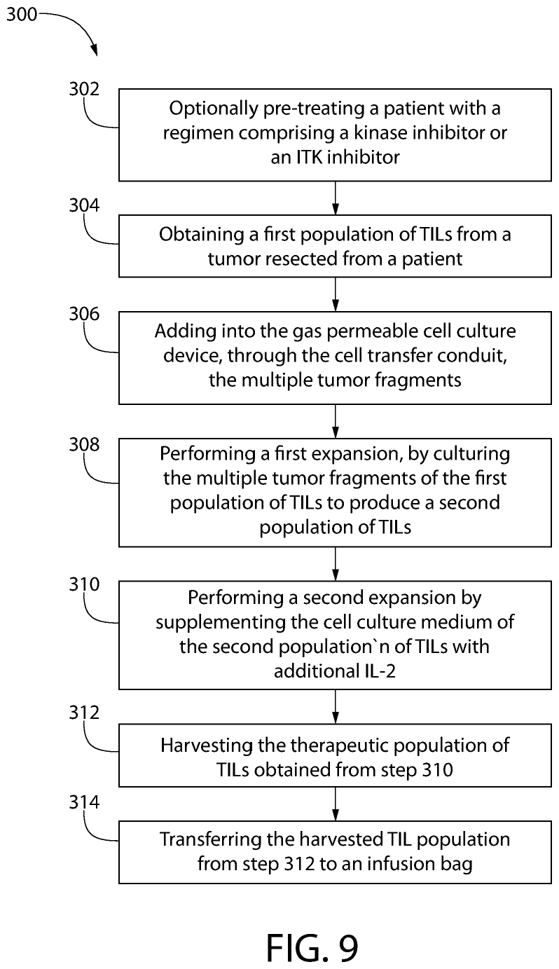

FIG. 9 illustrates an exemplary method 300 of using cell culture device 100 for expanding TILs into a therapeutic population of TILs. Method 300 may include step 302 of pre-treating a patient with a regimen comprising a kinase inhibitor or an ITK inhibitor. In some embodiments, step 302 is optionally performed. Method 300 may include step 304 of obtaining a first population of TILs from a tumor resected from a patient by dissecting a tumor sample resected from a patient into multiple tumor fragments. Method 300 may include step 306 of adding into the gas permeable cell culture device, through the cell transfer conduit, the multiple tumor fragments. Method 300 may further include step 308 of performing a first expansion, by culturing the multiple tumor fragments of the first population of TILs in a cell culture medium disposed within the gas permeable cell culture device. In some embodiments, the cell culture medium comprises IL-2 and optionally OKT-3. The first expansion may be performed to produce a second population of TILs during an expansion period of fourteen days or less after initiation of the first expansion. In some embodiment, the second population of TILs is at least 50-fold greater in number than the first population of TILs, and the only added source of gas to the expanding TIL population during the expansion period is through the gas permeable membrane.

In some embodiments, cell culture medium is provided in a container selected from the group consisting of a G-container and a Xuri cellbag. The cell culture medium may comprise IL-15 and/or IL-21. In some embodiments, the IL-2 concentration is from about 10,000 IU/mL to about 5,000 IU/mL. In some embodiments, the IL-15 concentration is from about 20 IU/mL to about 0.5 IU/mL. In some embodiments, the IL-21 concentration is from about 20 IU/mL to about 0.5 IU/mL.

In some embodiments, the multiple tumor fragments comprise about 4 to about 50 fragments and each fragment has a volume of about 27 mm³. However, the multiple tumor fragments may comprise from 2 to 200 fragments, 10 to 150 fragments, or 20 to 100 fragments. For example, the multiple tumor fragments may comprise about 30 to about 60 fragments with a total volume of about 1300 mm³ to about 1500 mm³ or may comprise about 50 fragments with a total volume of about 1350 mm³. The total volume of the fragments may be from 10 mm³ to 2000 mm³, 50 mm³ to 1500 mm³, 200 mm³ to 1300 mm³ or 500 mm³ to 1000 mm³. The multiple tumor fragments may comprise about 4 fragments. In some embodiments, the multiple tumor fragments comprise about 50 fragments with a total mass from about 1 gram to about 1.5 grams.

Referring to FIG. 9, method 300 may include step 310 of performing a second expansion by supplementing the cell culture medium of the second population of TILs with additional IL-2, optionally OKT-3, and antigen presenting cells (APCs). In some embodiments, the second expansion is performed to produce a third population of TILs. The second expansion may be performed for a second expansion period of fourteen days or less, wherein the third population of TILs is a therapeutic population of TILs. In some embodiments, the second expansion is performed in a second gas-permeable cell culture device. In some embodiments, the only added source of gas to the second expanding TIL population during the second expansion period is through a second gas permeable membrane in the second gas-permeable cell culture device. In some embodiments, the antigen-presenting cells are peripheral blood mononuclear cells (PBMCs). The PBMCs may be irradiated and allogenic. In some embodiments, the PBMCs are added to the cell culture on any of days 9 through 14 of the second expansion period. In some embodiments, the antigen-presenting cells are artificial antigen-presenting cells.

Method 300 may include step 312 of harvesting the therapeutic population of TILs obtained from step 310. In some embodiments, step 312 is performed using a membrane-based cell processing system. Step 312 may be performed using a LOVO cell processing system. Method 300 may also include step 314 of transferring the harvested TIL population from step 312 to an infusion bag. In some embodiments, steps 306 to 312 are performed within a closed system, such as cell culture device 100, wherein the only source of added gas to the closed system is through first and second gas permeable membranes. In some embodiments, method 300 may further include infusing the TILs from step 312 into a patient.

In some embodiments, the infusion bag is a HypoThermosol-containing infusion bag. In some embodiments, the therapeutic population of TILs harvested in step 312 comprises sufficient TILs for a therapeutically effective dosage of TILs. For example, the number of TILs sufficient for a therapeutically effective dosage may be from about 2.3×10¹⁰ to about 13.7×10¹⁰. 70. In some embodiments, the antigen-presenting cells are added to the TILs during the second period in step 312 without opening the system.

In some embodiments, the third population of TILs in step 312 provides for increased efficacy, increased interferon-gamma production, increased polyclonality, increased average IP-10, and/or increased average MCP-1 when administered to a subject. In some embodiments, the third population of TILs in step 312 provides for at least a five-fold or more interferon-gamma production when administered to a subject.

In some embodiments, the third population of TILs in step 312 is a therapeutic population of TILs that comprises an increased subpopulation of effector T cells and/or central memory T cells relative to the second population of TILs. The effector T cells and/or central memory T cells in the therapeutic population of TILs may exhibit one or more characteristics including expressing CD27+, expressing CD28+, longer telomeres, increased CD57 expression, and decreased CD56 expression relative to effector T cells, and/or central memory T cells obtained from the second population of cells. In some embodiments, the effector T cells and/or central memory T cells obtained from the third population of TILs exhibit increased CD57 expression and decreased CD56 expression relative to effector T cells and/or central memory T cells obtained from the second population of cells.

Method 300 may further comprise the step of cryopreserving the harvested TIL population in the infusion bag using a cryopreservation process. In some embodiments, the cryopreservation process is performed using a 1:1 ratio of harvested TIL population to cryopreservation media. In some embodiments, the cryopreservation media comprises dimethlysulfoxide (DMSO). The cryopreservation media may comprise 7% to 10% DMSO.

In some embodiments, the first period in step 308 and the second period in step 310 are each individually performed within a period of 10 days, 11 days, or 12 days. In some embodiments, first period in step 308 and the second period in step 310 are each individually performed within a period of 11 days.

In some embodiments, steps 304 through 314 are performed within a period of about 10 days to about 22 days. In some embodiments, steps 304 through 314 are performed within a period of about 20 days to about 22 days. In some embodiments, steps 304 through 314 are performed within a period of about 15 days to about 20 days. In some embodiments, steps 304 through 314 are performed within a period of about 10 days to about 20 days. In some embodiments, steps 304 through 314 are performed within a period of about 10 days to about 15 days. In some embodiments, steps 304 through 314 are performed in 22 days or less. In some embodiments, steps 304 through 314 are performed in 20 days or less. In some embodiments, steps 304 through 314 are performed in 15 days or less. In some embodiments, steps 304 through 314 are performed in 10 days or less. In some embodiments, steps 304 through 314 or steps 304 through 312 and cryopreservation are performed in 22 days or less.

In some embodiments, steps 306 through 312 are performed in a single container. Performing steps 306 through 312 in a single container may result in an increase in TIL yield per resected tumor as compared to performing steps 306 through 312 in more than one container, such as container 102. In some embodiments, performing method 300 in a closed system, such as cell culture device 100, reduces of the risk of microbial contamination compared to an open system.

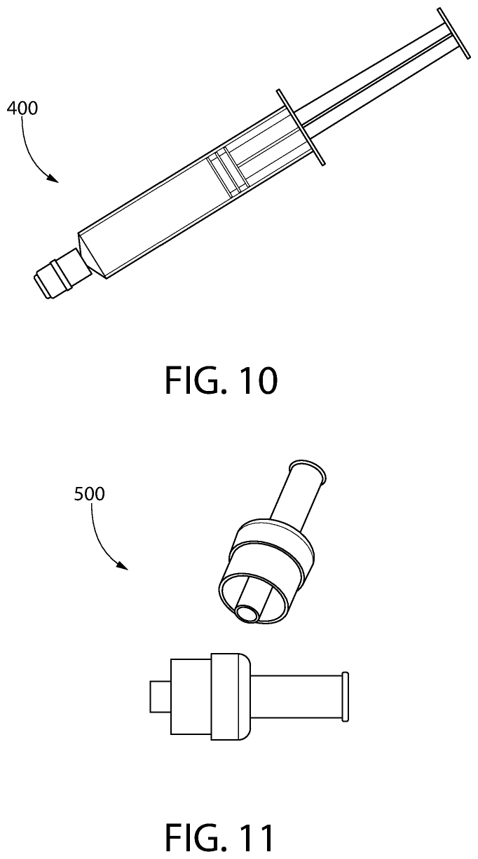

Referring to FIGS. 10-13 and 15, syringe 400 may be used to inject IL-2 into a bag or container of cell culture media, such as cell culture device 100. Syringe 400 may be a single use syringe containing IL-2 at a volume and biological activity sufficient to continuously expand T-cells for a predetermined amount of time. The predetermined amount of time may be from 5 days to 11 days. In some embodiments, syringe 400 is used to inject IL-2 into a bag of cell culture media via luer lock 500. Luer lock 500 may be a connection to a bioreactor suitable for expansion of T-cells. In some embodiments, syringe 400 is used to inject IL-2 into a bag of cell culture media via adapter 600. Adapter 600 may be configured to allow for needless injection into a bag of cell culture media. Adapter 600 may provide a needleless injection site or other syringe coupler to a bioreactor suitable for expansion of T-cells. Syringe 400 may be used to inject IL-2 into a bag of cell culture media through a connection made by a sterile connecting device.

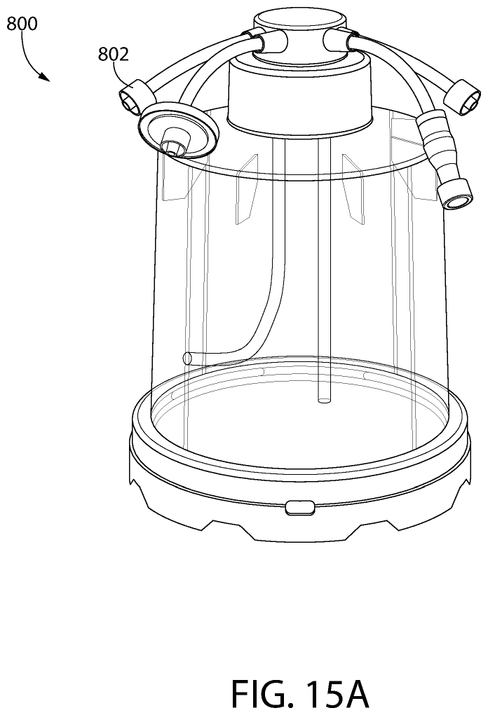

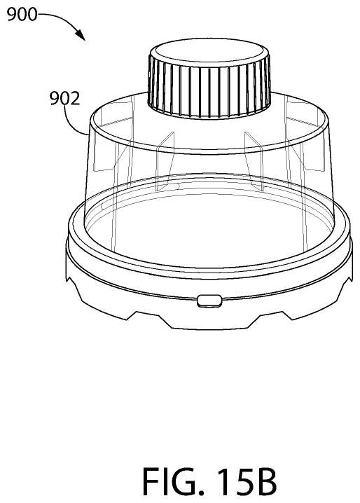

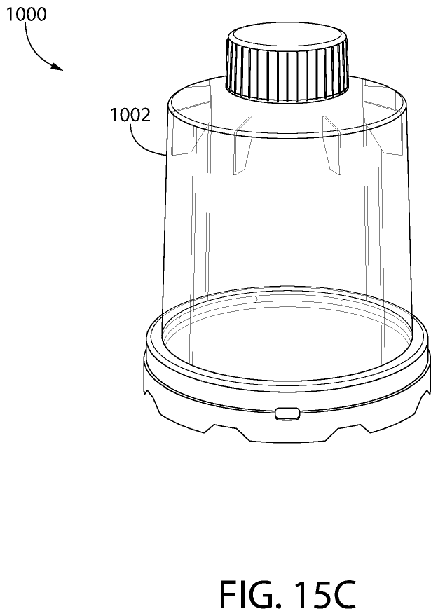

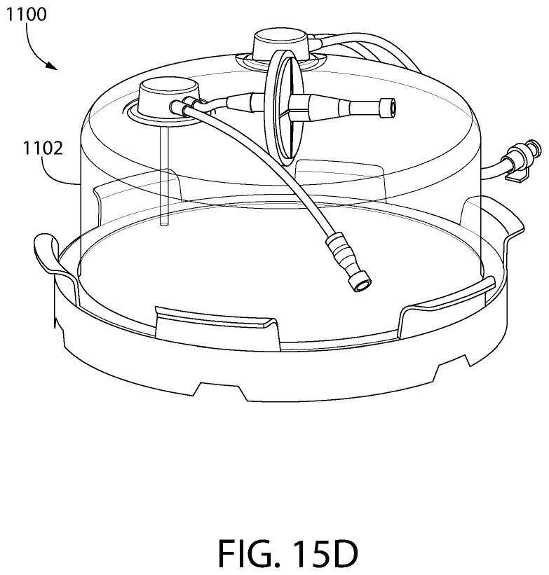

In some embodiments, the sterile connecting device may be used to inject IL-2 into a bioreactor suitable for expansion of T-cells. In some embodiments, syringe 400 is used to inject IL-2 into a bag or container of cell culture media, such as cell culture device 100, via connector 702. Connector 702 may be a weldable tubing. In some embodiments, the cell culture media is subsequently added to a bioreactor suitable for expansion of T-cells. (e.g., after the media is combined with IL-2). In some embodiments, syringe 400, luer lock 500, adapter 600, and/or connector 702 may be used with one or more containers 800, 900, 1000, and 1100 (illustrated in FIGS. 15A-15D).

In some embodiments, containers 800, 900, 1000, and 1100 are GREX container produced containers sold by WilsonWolf. For example, IL-2 may be added to container one or more of containers 800, 900, 1000, and 1100 for culturing of TILs. For example, one or more of syringe 400, luer lock 500, adapter 600, and/or connector 702 may be used to deliver IL-2 to container one or more containers 800, 900, 1000, 1100 via one or more of adapter adapters 802, 902, 1002, 1102.

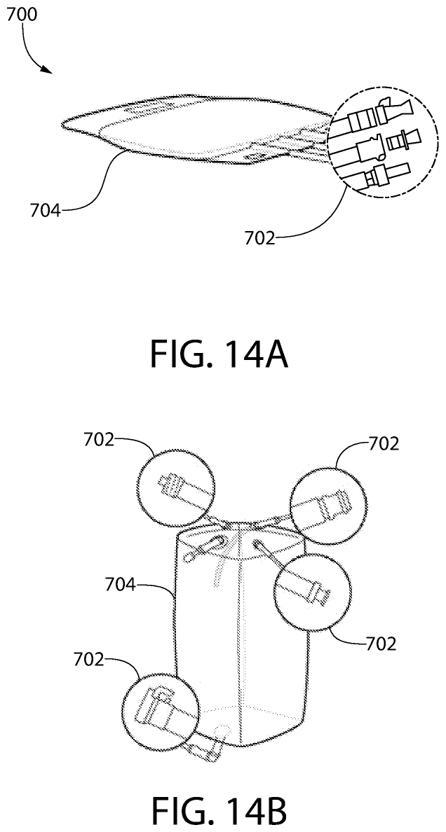

Referring to FIGS. 14A-15, container 700 may contain IL-2 at a volume and biological activity to continuously expand T-cells for a predetermined amount of time. The predetermined amount of time may be from 5 days to 11 days. Container 700 may be connected to a source of cell culture media, such as cell culture device 100, via a luer lock, such as luer lock 500, and may be pumped or gravity drained into the cell culture media. In some embodiments, the media is subsequently added to a bioreactor suitable for expansion of T-cells. In some embodiments, container 700 is coupled to a bag of cell culture media via a needless injection site, such as adapter 600, or another syringe coupling device. In some embodiments, container 700 is coupled to a source or bag of cell culture media, such as cell culture device 100, via the needless injection site or syringe coupling device and is pumped or gravity drained into a bioreactor suitable for expansion of T-cells. In some embodiments, container 700 containing IL-2 at a volume and biological activity sufficient to continuously expand T-cells is connected and pumped or gravity drained into a bioreactor suitable for expansion of T-cells through a connection made by a sterile connecting device. In some embodiments, container 700 is coupled to a bag of cell culture media via a sterile connecting device. Container 700 containing IL-2 may be pumped or gravity drained into the cell culture media. In some embodiments, the media is subsequently added to a bioreactor suitable for expansion of T-cells.

In some embodiments, container 700 is a 2D or 3D collapsible container. Container 700 may include a plurality of connectors/adapters 702. Connector 702 may be extending from container 700 and may be configured to couple to other storage or delivery devices, such as syringe 400. Container 700 may include body 704, which may be configured to expand and collapse based on the volume of media stored within. In some embodiments, container 700 has a collapsed state (FIG. 14A) and an expanded state (FIG. 14B).

In some embodiments, container 700 may be used with container 800. In some embodiments, container 800 is a GREX900, container produced by WilsonWolf. Container 800 may be substantially similar to container 102 of cell culture device 100. In some embodiments, IL-2 is added to container 800 for culturing of TILs via container 700.1000, and/or container 1100. In some embodiments, containers 800, 900, 1000, 1100 are GREX containers sold by WilsonWolf. In practice, IL-2 may be added to one or more of containers 800, 900, 1000, or 1100 for culturing of TILs via container 700. In some embodiments, each of containers 800, 900, 1000, or 1100 are substantially similar to container 102 and are used with cell culture device 100.

In some embodiments, one or more of containers 102, 700, 800, 900, 1000, 1100 are configured, as described herein, to receive material in the interior of the container without opening the container or exposing the interior of the container to atmosphere, either directly or indirectly, similar to container 102 of cell culture device 100. In one aspect of the invention, the method disclosed in PCT/US2019/031624 for treating cells and growing media is undertaken in one or more of containers 102, 700, 800, 900, 1000, or 1100. For example, one or more of containers 102, 700, 800, 900, 1000, 1100 may be used for a method of expanding TILs into a therapeutic population of TILs as disclosed in WO2019217753A1, which is hereby incorporated by reference in its entirety.

In some embodiments, the present disclosure provides methods of expanding TILs using the containers/devices described herein for treating a subject with cancer, the method comprising:
(a) obtaining a first population of TILs from a tumor resected from the subject by processing a tumor sample obtained from the subject into multiple tumor fragments;
(b) adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, wherein the second population of TILs is at least 50-fold greater in number than the first population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs which comprises an increased subpopulation of effector T cells and/or central memory T cells relative to the second population of TILs, wherein the second expansion is performed in a closed container providing a second gas-permeable surface area, and wherein the transition from step (c) to step (d) occurs without opening the system;
(e) harvesting the therapeutic population of TILs obtained from step (d), wherein the transition from step (d) to step (e) occurs without opening the system; and
(f) transferring the harvested TIL population from step (e) to an infusion bag, wherein the transfer from step (e) to (f) occurs without opening the system;
(g) optionally cryopreserving the infusion bag comprising the harvested TIL population from step (f) using a cryopreservation process; and
(h) administering a therapeutically effective dosage of the third population of TILs from the infusion bag in step (g) to the subject.

In some embodiments, the invention provides a therapeutic population of tumor infiltrating lymphocytes (TILs) expanded using the containers/devices described herein for use in treating cancer, wherein the population is obtainable from a method comprising the steps of:
(b) adding tumor fragments into a closed system wherein the tumour fragments comprise a first population of TILs;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, wherein the second population of TILs is at least 50-fold greater in number than the first population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs which comprises an increased subpopulation of effector T cells and/or central memory T cells relative to the second population of TILs, wherein the second expansion is performed in a closed container providing a second gas-permeable surface area, and wherein the transition from step (c) to step (d) occurs without opening the system;
(e) harvesting the therapeutic population of TILs obtained from step (d), wherein the transition from step (d) to step (e) occurs without opening the system; and
(f) transferring the harvested TIL population from step (e) to an infusion bag, wherein the transfer from step (e) to (f) occurs without opening the system; and
(g) optionally cryopreserving the infusion bag comprising the harvested TIL population from step (f) using a cryopreservation process.

In some embodiments, the population is obtainable by a method also comprising as a first step:
(a) obtaining a first population of TILs from a tumor resected from a subject by processing a tumor sample obtained from the subject into multiple tumor fragments.

In an embodiment, the method is an in vitro or an ex vivo method.

In some embodiments, any of steps (a) to (f) comprise one or more features disclosed herein, e.g. one or more features disclosed under the headings “STEP A: Obtain Subject Tumor Sample”, “STEP B: First Expansion”, “STEP C: First Expansion to Second Expansion Transition”, “STEP D: Second Expansion”, “STEP E: Harvest TILs and “STEP F: Final Formulation/Transfer to Infusion Bag”.

In some embodiments, step (g) comprises one or more features disclosed herein, e.g. one or more features disclosed under the heading “STEP H: Optional Cryopreservation of TILs”. In some embodiments, step (h) comprise one or more features disclosed herein, e.g. one or more features disclosed under the heading “STEP F:1 Pharmaceutical Compositions, Dosages and Dosing Regimens”.

In some embodiments, the therapeutic population of TILs harvested in step (e) comprises sufficient TILs for administering a therapeutically effective dosage of the TILs in step (h).

In some embodiments, the number of TILs sufficient for administering a therapeutically effective dosage in step (h) is from about 2.3×10¹⁰ to about 13.7×10¹⁰.

In some embodiments, the antigen presenting cells (APCs) are PBMCs.

In some embodiments, the PBMCs are added to the cell culture on any of days 9 through 14 in step (d).

In some embodiments, prior to administering a therapeutically effective dosage of TIL cells in step (h), a non-myeloablative lymphodepletion regimen has been administered to the subject.

In some embodiments, there is provided a therapeutic population of tumor infiltrating lymphocytes (TILs) expanded using the containers/devices described herein for use in treating cancer and in combination with a non-myeloablative lymphodepletion regimen. In some embodiments, the non-myeloablative lymphodepletion regimen is administered prior to administering the therapeutic population of tumor infiltrating lymphocytes (TILs).

In some embodiments, the non-myeloablative lymphodepletion regimen comprises the steps of administration of cyclophosphamide at a dose of 60 mg/m²/day for two days followed by administration of fludarabine at a dose of 25 mg/m²/day for five days.

In some embodiments, the step of treating the subject with a high-dose IL-2 regimen starting on the day after administration of the TIL cells to the subject in step (h).

In some embodiments, there is provided a therapeutic population of tumor infiltrating lymphocytes (TILs) expanded using the containers/devices described herein for use in treating cancer and in combination with high-dose IL-2 regimen. In some embodiments, the high-dose IL-2 regimen starts on the day after administration of the therapeutic population of TIL cells.

In some embodiments, the high-dose IL-2 regimen comprises 600,000 or 720,000 IU/kg administered as a 15-minute bolus intravenous infusion every eight hours until tolerance.

In some embodiments, the effector T cells and/or central memory T cells in the therapeutic population of TILs exhibit one or more characteristics selected from the group consisting of expressing CD27+, expressing CD28+, longer telomeres, increased CD57 expression, and decreased CD56 expression relative to effector T cells, and/or central memory T cells obtained from the second population of cells.

In some embodiments, the effector T cells and/or central memory T cells in the therapeutic population of TILs exhibit increased CD57 expression and decreased CD56 expression relative to effector T cells and/or central memory T cells obtained from the second population of cells.

The present disclosure also provides methods for expanding tumor infiltrating lymphocytes (TILs) using the containers/devices described herein into a therapeutic population of TILs comprising:
(a) adding processed tumor fragments from a tumor resected from a subject into a closed system to obtain a first population of TILs;
(b) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, wherein the second population of TILs is at least 50-fold greater in number than the first population of TILs, and wherein the transition from step (a) to step (b) occurs without opening the system;
(c) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs which comprises an increased subpopulation of effector T cells and/or central memory T cells relative to the second population of TILs, wherein the second expansion is performed in a closed container providing a second gas-permeable surface area, and wherein the transition from step (b) to step (c) occurs without opening the system;
(d) harvesting the therapeutic population of TILs obtained from step (c), wherein the transition from step (c) to step (d) occurs without opening the system; and
(e) transferring the harvested TIL population from step (d) to an infusion bag, wherein the transfer from step (d) to (e) occurs without opening the system.

In some embodiments, the therapeutic population of TILs harvested in step (d) comprises sufficient TILs for a therapeutically effective dosage of the TILs.

In some embodiments, the number of TILs sufficient for a therapeutically effective dosage is from about 2.3×10¹⁰ to about 13.7×10¹⁰.

In some embodiments, the method further comprises the step of cryopreserving the infusion bag comprising the harvested TIL population using a cryopreservation process.

In some embodiments, the cryopreservation process is performed using a 1:1 ratio of harvested TIL population to CS10 media.

In some embodiments, the present disclosure provides methods for treating a subject with cancer, the method comprising administering tumor infiltrating lymphocytes (TILs) expanded using the containers/devices described herein comprising:
(a) obtaining a first population of TILs from a tumor resected from the subject by processing a tumor sample obtained from the subject into multiple tumor fragments;
(b) adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, wherein the second population of TILs is at least 50-fold greater in number than the first population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs which comprises an increased subpopulation of effector T cells and/or central memory T cells relative to the second population of TILs, wherein the second expansion is performed in a closed container providing a second gas-permeable surface area, and wherein the transition from step (c) to step (d) occurs without opening the system;
(e) harvesting the therapeutic population of TILs obtained from step (d), wherein the transition from step (d) to step (e) occurs without opening the system; and
(f) transferring the harvested TIL population from step (e) to an infusion bag, wherein the transfer from step (e) to (f) occurs without opening the system;
(g) optionally cryopreserving the infusion bag comprising the harvested TIL population from step (f) using a cryopreservation process; and
(h) administering a therapeutically effective dosage of the third population of TILs from the infusion bag in step (g) to the subject.
   wherein no selection of TIL population is performed during any of steps (a) to (h). In an embodiment, no selection of the second population of TILs (the pre-REP population) based on phenotype is performed prior to performing the second expansion of step (d). In an embodiment, no selection of the first population of TILs, second population of TILs, third population of TILs, or harvested TIL population based on CD8 expression is performed during any of steps (a) to (h).

In some embodiments, the present disclosure provides methods for treating a subject with cancer, the method comprising administering tumor infiltrating lymphocytes (TILs) expanded using the containers/devices described herein comprising:
(a) obtaining a first population of TILs from a tumor resected from the subject by processing a tumor sample obtained from the subject into multiple tumor fragments;
(b) adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, wherein the second population of TILs is at least 50-fold greater in number than the first population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs which comprises an increased subpopulation of effector T cells and/or central memory T cells relative to the second population of TILs, wherein the second expansion is performed in a closed container providing a second gas-permeable surface area, and wherein the transition from step (c) to step (d) occurs without opening the system;
(e) harvesting the therapeutic population of TILs obtained from step (d), wherein the transition from step (d) to step (e) occurs without opening the system; and
(f) transferring the harvested TIL population from step (e) to an infusion bag, wherein the transfer from step (e) to (f) occurs without opening the system;
(g) cryopreserving the infusion bag comprising the harvested TIL population from step (f) using a cryopreservation process, wherein the cryopreservation process comprises mixing of a cryopreservation media with the harvested TIL population;
(h) administering a therapeutically effective dosage of the third population of TILs from the infusion bag in step (g) to the subject.
   wherein no selection of TIL population is performed during any of steps (a) to (h). In an embodiment, no selection of the second population of TILs (for example, the pre-REP population) based on phenotype is performed prior to performing the second expansion of step (d). In an embodiment, no selection of the first population of TILs, second population of TILs, third population of TILs, or harvested TIL population based on CD8 expression is performed during any of steps (a) to (h). In some embodiments, the non-myeloablative lymphodepletion regimen is administered prior to administering the harvested TIL population. In some embodiments, the non-myeloablative lymphodepletion regimen comprises the steps of administration of cyclophosphamide at a dose of 60 mg/m²/day for two days followed by administration of fludarabine at a dose of 25 mg/m²/day for five days.

In some embodiments, the antigen-presenting cells are peripheral blood mononuclear cells (PBMCs). In some embodiments, the PBMCs are irradiated and allogeneic. In some embodiments, the PBMCs are added to the cell culture on any of days 9 through 14 in step (c).

In some embodiments, the antigen-presenting cells are artificial antigen-presenting cells.

In some embodiments, the harvesting in step (d) is performed using a LOVO cell processing system.

In some embodiments, the method comprises harvesting in step (d) is via a LOVO cell processing system, such as the LOVO system manufactured by Fresenius Kabi. The term “LOVO cell processing system” also refers to any instrument or device that can pump a solution comprising cells through a membrane or filter such as a spinning membrane or spinning filter in a sterile and/or closed system environment, allowing for continuous flow and cell processing to remove supernatant or cell culture media without pelletization. In some cases, the cell processing system can perform cell separation, washing, fluid-exchange, concentration, and/or other cell processing steps in a closed, sterile system.

In some embodiments, the tumor fragments are multiple fragments and comprise about 4 to about 50 fragments, wherein each fragment has a volume of about 27 mm³. In some embodiments, the multiple fragments comprise about 30 to about 60 fragments with a total volume of about 1300 mm³ to about 1500 mm³. In some embodiments, the multiple fragments comprise about 50 fragments with a total volume of about 1350 mm³. In some embodiments, the multiple fragments comprise about 50 fragments with a total mass of about 1 gram to about 1.5 grams.

In some embodiments, the multiple fragments comprise about 4 fragments. In some embodiments, the 4 fragments are placed into a G-REX-100. In some embodiments, the 4 fragments are about 0.1 cm, 0.2 cm, 0.3 cm, 0.4 cm, 0.5 cm, 0.6 cm, 0.7 cm, 0.8 cm, 0.9 cm, or 1 cm in diameter. In some embodiments, the 4 fragments are about 0.1 cm, 0.2 cm, 0.3 cm, 0.4 cm, 0.5 cm, 0.6 cm, 0.7 cm, 0.8 cm, 0.9 cm, or 1 cm in diameter and are placed into a G-REX-100. In some embodiments, the 4 fragments are about 0.1 cm, 0.2 cm, 0.3 cm, 0.4 cm, 0.5 cm, 0.6 cm, 0.7 cm, 0.8 cm, 0.9 cm, or 1 cm in diameter are placed into a container with an equivalent volume to a G-REX-100. In some embodiments, the 4 fragments are about 0.5 cm in diameter and are placed into a G-REX-100. In some embodiments, the 4 fragments are about 0.5 cm in diameter and are placed into a container with an equivalent volume to a G-REX-100.

In some embodiments, the cell culture medium is provided in a container selected from the group consisting of a G-container and a Xuri cellbag.

In some embodiments, the infusion bag in step (e) is a HypoThermosol-containing infusion bag.

In some embodiments, the first period in step (b) and the second period in step (c) are each individually performed within a period of 10 days, 11 days, or 12 days. In some embodiments, the first period in step (b) and the second period in step (c) are each individually performed within a period of 11 days. In some embodiments, steps (a) through (e) are performed within a period of about 25 days to about 30 days. In some embodiments, steps (a) through (e) are performed within a period of about 20 days to about 25 days. In some embodiments, steps (a) through (e) are performed within a period of about 20 days to about 22 days. In some embodiments, steps (a) through (e) are performed in 22 days or less. In some embodiments, steps (a) through (e) and cryopreservation are performed in 22 days or less.

In some embodiments, steps (b) through (e) are performed in a single container, wherein performing steps (b) through (e) in a single container results in an increase in TIL yield per resected tumor as compared to performing steps (b) through (e) in more than one container.

In some embodiments, the antigen-presenting cells are added to the TILs during the second period in step (c) without opening the system.

In some embodiments, the effector T cells and/or central memory T cells obtained in the therapeutic population of TILs exhibit one or more characteristics selected from the group consisting of expressing CD27+, expressing CD28+, longer telomeres, increased CD57 expression, and decreased CD56 expression relative to effector T cells, and/or central memory T cells obtained from the second population of cells.

In some embodiments, the effector T cells and/or central memory T cells obtained in the therapeutic population of TILs exhibit increased CD57 expression and decreased CD56 expression relative to effector T cells, and/or central memory T cells obtained from the second population of cells.

In some embodiments, the risk of microbial contamination is reduced as compared to an open system.

In some embodiments, the TILs from step (e) are infused into the subject.

In some embodiments, the closed container comprises a single bioreactor. In some embodiments, the closed container comprises a G-REX-10. In some embodiments, the closed container comprises a G-REX-100.

In some embodiments, the subject is a mammal. In some embodiments, the subject is a human. In some embodiments, the subject is a patient.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations were chosen in order to best explain the principles underlying the claims and their practical applications, to thereby enable others skilled in the art to best use the implementations with various modifications as are suited to the particular uses contemplated.

1. A gas permeable cell culture device comprising:
a container body defining an interior volume, the container body having a semi-permeable membrane defining one end of the interior volume;
a fitting defining an opposing end of the interior volume; and
a cell transfer conduit having
an open end positioned within the interior volume between the semi-permeable membrane and the fitting; and
a radial portion disposed within the interior volume and between the open end and opposing end of the container body.

2. The gas permeable cell culture device of claim 1 wherein the interior volume is configured to include a headspace and at least a portion of the radial portion is disposed in the headspace.

3. The gas permeable cell culture device of claim 1 wherein the radial portion comprises a segment of the cell transfer conduit having a longitudinal axis with at least one finite radius of curvature.

4. The gas permeable cell culture device of claim 1 wherein the cell transfer conduit has an internal diameter of approximately 0.125 inches.

5. The gas permeable cell culture device of claim 1 wherein the cell transfer conduit includes an interior portion extending from the fitting to the open end, the interior portion having an inner wall radially disposed about a longitudinal axis wherein no segment of the longitudinal axis of the interior portion has a radius of curvature that is less than 30 centimeters.

6. The gas permeable cell culture device of claim 1 wherein the cell transfer conduit includes a transfer portion in fluid communication with the radial portion and extending from the fitting.

7. The gas permeable cell culture device of claim 6 wherein the fitting includes a plurality of apertures, at least one of the plurality of apertures configured to receive the transfer portion of the cell transfer conduit.

8. The gas permeable cell culture device of claim 1 further comprising a gas inlet conduit extending through the fitting.

9. The gas permeable cell culture device of claim 2 further comprising an extraction conduit extending into the head space through the fitting.

10. The gas permeable cell culture device of claim 2 further comprising a feeding conduit extending into the head space through the fitting.

11. The gas permeable cell culture device of claim 10 wherein the feeding conduit includes a transfer portion extending through the fitting.

12. The gas permeable cell culture device of claim 8 wherein the gas inlet conduit includes a transfer portion extending through the fitting.

13. The gas permeable cell culture device of claim 1 further comprising machine readable indicia disposed on a surface of the gas permeable cell culture device.

14. The gas permeable cell culture device of claim 13 wherein the machine readable indicia is readable to indicate at least one of the parameters selected from the group consisting of: 1) information associated with an origin of cells disposed within the cell culture device; 2) time information associated with injection and/or extraction of cells with respect to the cell culture device; 3) information with respect to media disposed within or extracted from the cell culture device; 4) information associated with a patient donor of the cells disposed within the cell culture device; 5) identification and tracking of a chain of custody and/or chain of identity associated with cell culture device and/or the cells disposed within the cell culture device; 6) information associated with quality control data; 7) information associated with electronic batch records; 8) information associated with manufacturing database and/or enterprise resource planning; and 9) combinations thereof.

15. The gas permeable cell culture device of claim 13 wherein the machine readable indicia is selected from a group consisting of bar coding, QR coding, RFID, magnetic strips, one or more photographs or text.

16. The gas permeable cell culture device of claim 1 wherein the cell transfer conduit passes through a vertical sidewall of the fitting.

17. The gas permeable cell culture device of claim 1 further comprising at least one conduit passing through a horizontal top wall of the fitting and the cell transfer conduit passing through a vertical sidewall of the fitting.

18. The gas permeable cell culture device of claim 1 further comprising a separation membrane disposed between the fitting and the interior volume of the container.

19. The gas permeable cell culture device of claim 18 wherein the separation membrane includes a plurality of apertures, at least one of the plurality of apertures configured to receive a portion of the cell transfer conduit.

20. The gas permeable cell culture device of claim 19 wherein the separation membrane is coupled to the fitting.

21. The gas permeable cell culture device of claim 1 wherein the fitting is removably coupled to the container.

22. The gas permeable cell culture device of claim 1 wherein the fitting is fixedly coupled to the container.

23. The gas permeable cell culture device of claim 1 wherein the cell transfer conduit includes at least one attachment piece.

24. The gas permeable cell culture device of claim 8 wherein the gas inlet conduit is coupled to a sterile filter sized between 0.05 micrometers and 0.25 micrometers.

25. The gas permeable cell culture device of claim 1 further comprising a bottom fitting coupled to a bottom of the container.

26. The gas permeable cell culture device of claim 25 wherein the bottom fitting is adjacent to the semi-permeable membrane.

27. The gas permeable cell culture device of claim 26 wherein the bottom fitting is configured to be removably coupled to a tray.

28. The gas permeable cell culture device of claim 27 wherein the tray is configured to receive a plurality of bottom fittings.

29. The gas permeable cell culture device of claim 1 wherein the cell transfer conduit is configured to be heat welded to seal the cell transfer conduit.

30. The gas permeable cell culture device of claim 8 wherein the gas inlet conduit includes a radial portion disposed within the interior volume and between the open end and opposing end of the container body.

31. The gas permeable cell culture device of claim 1 further comprising a gas inlet conduit, a extraction conduit, and a feeding conduit, wherein any of the gas inlet conduit, the extraction conduit, or the feeding conduit includes a radial portion disposed within the interior volume and between the open end and opposing end of the container body.

32. The gas permeable cell culture device of claim 1 wherein a ratio of a volume of the container to a surface area of the semi-permeable membrane is between 1 mL/cm² and 10 mL/cm².

33. A method of using the gas permeable cell culture device of claim 1 for expanding tumor infiltrating lymphocytes (TILs) into a therapeutic population of TILs, the method comprising:
(a) optionally pre-treating a patient with a regimen comprising a kinase inhibitor or an ITK inhibitor;
(b) obtaining a first population of TILs from a tumor resected from a patient by dissecting a tumor sample resected from a patient into multiple tumor fragments;
(c) adding into the gas permeable cell culture device, through the cell transfer conduit, the multiple tumor fragments;
(d) performing a first expansion, by culturing the multiple tumor fragments of the first population of TILs in a cell culture medium disposed within the gas permeable cell culture device, the cell culture medium comprising IL-2, and optionally OKT-3, to produce a second population of TILs during an expansion period of fourteen days or less after initiation of the first expansion, wherein the second population of TILs is at least 50-fold greater in number than the first population of TILs, and the only added source of gas to the expanding TIL population during the expansion period is through the semi-permeable membrane;
(e) performing a second expansion by supplementing the cell culture medium of the second population of TILs with additional IL-2, optionally OKT-3, and antigen presenting cells (APCs), to produce a third population of TILs, wherein the second expansion is performed for a second expansion period of fourteen days or less, wherein the third population of TILs is a therapeutic population of TTLs, wherein the second expansion is performed in a second gas-permeable cell culture device and the only added source of gas to the second expanding TIL population during the second expansion period is through a second gas permeable membrane in the second gas-permeable cell culture device;
(f) harvesting the therapeutic population of TILs obtained from step (e); and
(g) transferring the harvested TIL population from step (f) to an infusion bag.

34. The method of step 33 wherein steps (c)-(f) are performed within a closed system wherein the only source of added gas to the closed system is through first and second gas permeable membranes.

35. The method of claim 33, further comprising the step of cryopreserving the harvested TIL population in the infusion bag using a cryopreservation process.

36. The method of claim 35, wherein the cryopreservation process is performed using a 1:1 ratio of harvested TIL population to cryopreservation media.

37. The method of claim 33, wherein the antigen-presenting cells are peripheral blood mononuclear cells (PBMCs).

38. The method of claim 37, wherein the PBMCs are irradiated and allogeneic.

39. The method of claim 38, wherein the PBMCs are added to the cell culture on any of days 9 through 14 of the second expansion period.

40. The method of claim 33, wherein the antigen-presenting cells are artificial antigen-presenting cells.

41. The method of claim 33, wherein the harvesting is performed using a membrane-based cell processing system.

42. The method of claim 33, wherein the harvesting in step (f) is performed using a LOVO cell processing system.

43. The method of claim 33, wherein the multiple tumor fragments comprise about 4 to about 50 fragments, wherein each fragment has a volume of about 27 mm³.

44. The method of claim 33, wherein the multiple tumor fragments comprise about 30 to about 60 fragments with a total volume of about 1300 mm³ to about 1500 mm³.

45. The method of claim 43, wherein the multiple tumor fragments comprise about 50 fragments with a total volume of about 1350 mm³.

46. The method of claim 33, wherein the multiple tumor fragments comprise about 50 fragments with a total mass of about 1 gram to about 1.5 grams.

47. The method of claim 33, wherein the cell culture medium is provided in a container selected from the group consisting of a G-container and a Xuri cellbag.

48. The method of claim 33, wherein the cell culture medium in step (d) further comprises IL-15 and/or IL-21.

49. The method of claim 33, wherein the IL-2 concentration is about 10,000 IU/mL to about 5,000 IU/mL.

50. The method of claim 33, wherein the IL-15 concentration is about 500 IU/mL to about 100 IU/mL.

51. The method of claim 33, wherein the IL-21 concentration is about 20 IU/mL to about 0.5 IU/mL.

52. The method of claim 33, wherein the infusion bag in step (g) is a Hypo(Original) Thermosol-containing infusion bag.

53. The method of claim 36, wherein the cryopreservation media comprises dimethlysulfoxide (DMSO).

54. The method of claim 36, wherein the cryopreservation media comprises 7% to 10% DMSO.

55. The method of claim 33, wherein the first expansion period in step (d) and the second expansion period in step (f) are each individually performed within a period of 10 days, 11 days, or 12 days.

56. The method of claim 33, wherein the first expansion period in step (d) and the second expansion period in step (f) are each individually performed within a period of 11 days.

57. The method of claim 33, wherein steps (b) through (g) are performed within a period of about 10 days to about 22 days.

58. The method of claim 33, wherein steps (b) through (g) are performed within a period of about 20 days to about 22 days.

59. The method of claim 33, wherein steps (b) through (g) are performed within a period of about 15 days to about 20 days.

60. The method of claim 33, wherein steps (b) through (g) are performed within a period of about 10 days to about 20 days.

61. The method of claim 33, wherein steps (b) through (g) are performed within a period of about 10 days to about 15 days.

62. The method of claim 33, wherein steps (b) through (g) are performed in 22 days or less.

63. The method of claim 33, wherein steps (b) through (g) are performed in 20 days or less.

64. The method of claim 33, wherein steps (b) through (g) are performed in 15 days or less.

65. The method of claim 33, wherein steps (b) through (g) are performed in 10 days or less.

66. The method of claim 33, wherein steps (b) through (g) or steps (b) through (f) and cryopreservation are performed in 22 days or less.

67. The method of claim 33, wherein the therapeutic population of TTLs harvested in step (f) comprises sufficient TILs for a therapeutically effective dosage of the TTLs.

68. The method of claim 67, wherein the population of TILs sufficient for a therapeutically effective dosage is from about 2.3×10¹⁰ to about 13.7×10¹⁰.

69. The method of claim 33 wherein steps (c) through (f) are performed in a single container, wherein performing steps (c) through (f) in a single container results in an increase in TIL yield per resected tumor as compared to performing steps (c) through (f) in more than one container.

70. The method of claim 33, wherein the antigen-presenting cells are added to the TILs during the second period in step (e) without opening the cell culture device.

71. The method of claim 33, wherein the third population of TILs in step (e) provides for increased efficacy, increased interferon-gamma production, increased polyclonality, increased average IP-10, and/or increased average MCP-1 when administered to a subject.

72. The method of claim 33, wherein the third population of TILs in step (e) provides for at least a five-fold or more interferon-gamma production when administered to a subject.

73. The method of claim 33, wherein the third population of TILs in step (e) is a therapeutic population of TILs that comprises an increased subpopulation of effector T cells and/or central memory T cells relative to the second population of TILs, wherein the effector T cells and/or central memory T cells in the therapeutic population of TILs exhibit one or more characteristics selected from the group consisting of expressing CD27+, expressing CD28+, longer telomeres, increased CD57 expression, and decreased CD56 expression relative to effector T cells, and/or central memory T cells obtained from the second population of cells.

74. The method of claim 73, wherein the effector T cells and/or central memory T cells obtained from the third population of TILs exhibit increased CD57 expression and decreased CD56 expression relative to effector T cells and/or central memory T cells obtained from the second population of cells.

75. The method of claim 33, wherein a risk of microbial contamination is reduced when the cell culture device is a closed system compared to when the cell culture device is an open system.

76. The method of claim 33 further comprising infusing the TILs from step (g) into a patient.

77. The method of claim 33, wherein the multiple tumor fragments comprise about 4 fragments.

78. A cell culture device comprising:
a top end cap having a first aperture;
a bottom end cap having a second aperture aligned with the first aperture; and
one or more assemblies disposed between the top end cap and the bottom end cap, wherein each one of the one or more of assemblies includes a top support, a bottom support, and a membrane layer disposed between the top support and the bottom support.

79. The cell culture device of claim 78, further comprising a central bore disposed through a central axis of the one or more assemblies, wherein the central bore aligns with the first aperture and the second aperture.

80. The cell culture device of claim 78, wherein each one of the one or more assemblies is disposed on an offset plane.

81. The cell culture device of claim 78, wherein each one of the one or more assemblies includes a first end and a second end offset from the first end, the first end and second end being disposed along an outer perimeter of each one of the one or more assemblies.

82. The cell culture device of claim 81, wherein one of the one or more assemblies is configured to be stackable with another one of the one or more assemblies such that the first end of one of the one or more assemblies is aligned with the first end of another one of the one or more assemblies.

83. The cell culture device of claim 81, wherein a thickness of the first end is greater than a thickness of the second end.

84. The cell culture device of claim 78, wherein each one of the one or more assemblies is circular.

85. The cell culture device of claim 78, wherein the membrane layer of each one of the one or more assemblies is a media-impermeable membrane.

86. The cell culture device of claim 78, wherein the membrane layer of each one of the one or more assemblies is a gas permeable membrane.

87. The cell culture device of claim 78, wherein the membrane layer of each one of the one or more assemblies is comprised of one or more of polydimethylsiloxane, copolymers, polyolefins, flouropolymers, fluorinated ethylene propylene (FEP), polyvinylchloride (PVC), ethylene-vinyl acetate (EVA), and other polymers or copolymers.

88. The cell culture device of claim 78, wherein the membrane layer of each one of the one or more assemblies is comprised of polystyrene film.

89. The cell culture device of claim 78, wherein the membrane layer of each one of the one or more assemblies has a thickness between about 25 micrometers and 125 micrometers.

90. The cell culture device of claim 78, wherein the bottom support and/or the top support of each one of the one or more assemblies is comprised of polystyrene.

91. The cell culture device of claim 78, wherein the membrane layer is coupled to the top support via one of adhesive bonding, ultrasonic welding, or laser welding.

92. The cell culture device of claim 78, wherein the top support is coupled to the bottom support via one of adhesive bonding, ultrasonic welding, or laser welding.

93. The cell culture device of claim 78, further comprising a port formed between the top support and the bottom support to allow for gas exchange.

94. The cell culture device of claim 93, wherein the port is disposed on an outer perimeter of each one of the one or more assemblies.

95. The cell culture device of claim 78 further comprising a housing and a housing cap coupled to the housing, wherein the one or more assemblies are disposed within the housing.

96. The cell culture device of claim 95, wherein the housing cap includes one or more openings and one or more conduits disposed within the one or more openings, each one of the one or more conduits extending from the one or more openings of the housing cap into the housing.

97. The cell culture device of claim 96, wherein at least one of the one or more conduits is in a spiral configuration within the housing cap prior to extending within the housing.

98. The cell culture device of claim 97, wherein each one of the one or more conduits has a different length.

99. The cell culture device of claim 97, wherein at least one of the one or more conduits includes an elbow joint disposed within the housing cap.

100. A single use syringe comprising
IL-2 at a volume and biological activity sufficient to continuously expand T-cells for a predetermined period of time, the T-cells being injected into a source of cell culture media via an attachment device and the cell culture media is subsequently added to a bioreactor suitable for expansion of T-cells.

101. The single use syringe of claim 100, wherein the predetermined period of time is 5-11 days and the attachment device is a luer lock.

102. The single use syringe of claim 100, wherein the predetermined period of time is 5-11 days and the attachment device is a needleless injection site or other syringe coupler.

103. The single use syringe of claim 100, wherein the predetermined period of time is 5-11 days and the attachment device is a sterile connecting device.

104. The single use syringe of claim 100, wherein the predetermined period of time is 5-11 days and the attachment device is a luer lock connection to the bioreactor.

105. The single use syringe of claim 100, wherein the predetermined period of time is 5-11 days and the attachment device is a needeless injection site or other syringe coupler connection to the bioreactor.

106. The single use syringe of claim 100, wherein the predetermined period of time is 5-11 days and the attachment device is a sterile connecting device connection to the bioreactor.

107. The single use syringe of claim 100, wherein the predetermined period of time is 5-11 days and the attachment device is a luer lock and T-cells are pumped or gravity drained into the cell culture media.

108. The single use syringe of claim 100, wherein the predetermined period of time is 5-11 days and the attachment device is a needleless injection site or other syringe coupler and T-cells are pumped or gravity drained into the cell culture media.

109. The single use syringe of claim 100, wherein the predetermined period of time is 5-11 days and the attachment device is a sterile connecting device and T-cells are pumped or gravity drained into the cell culture media.

110. A collapsible container comprising IL-2 at a volume and biological activity sufficient to continuously expand T-cells for a predetermined amount of time, the container connected via an attachment device and pumped or gravity drained into a bioreactor suitable for expansion of T-cells.

111. The collapsible container of claim 110 wherein the predetermined amount of time is 5-11 days and the attachment device is a luer lock.

112. The collapsible container of claim 110 wherein the predetermined amount of time is 5-11 days and the attachment device is a needleless injection site or other sterile coupler.

113. The collapsible container of claim 110 wherein the predetermined amount of time is 5-11 days and the attachment device is a sterile connecting device.
